(19) United States
(12) Patent Application Publication
(10) Pub. No.: US 2016/0058650 A1
(43) Pub. Date: Mar. 3, 2016

(54) VISION CORRECTION INSTRUMENT AND METHOD OF CORRECTING VISION

(71) Applicants: GUOXIAN ZHANG, QINGDAO CITY (CN); YAN LI, QINGDAO CITY (CN)

(72) Inventors: GUOXIAN ZHANG, QINGDAO CITY (CN); YAN LI, QINGDAO CITY (CN)

(21) Appl. No.: 14/803,115

(22) Filed: Jul. 20, 2015

(30) Foreign Application Priority Data
Aug. 21, 2014 (CN) 201410422708.6

(51) Int. Cl. A61H 5/00; A61B 3/032

(52) U.S. Cl. CPC A61H 5/00; A61B 3/032

(57) ABSTRACT

A vision correction instrument includes a display device, a support frame and a keyboard. The display device is provided on the support frame; the display device and the keyboard are in wireless connection; a power button is provided on the display device; and a certain distance is provided between the display device and the keyboard. A method of correcting vision includes the following steps: starting the display device; inputting the numbers or letters shown on a screen through the keyboard and pressing an enter key; repeating the previous step operation; returning to an initial state; and closing the display device. The vision correction instrument makes the ciliary muscle of the eyes continuously constrict and relax to adjust the change of lens, alleviate eye fatigue, increase ciliary activity and achieve the purpose of treatment of myopia.

### BACKGROUND OF THE INVENTION

1.

### Field of the Invention

The invention belongs to the field of vision correction, and specifically relates to a vision correction instrument and a method of correcting vision.

2. Description of Related Art

In recent years, China has an increased dramatically myopia trend, severely affecting a child's eye health. Human eyes are normal organs with far vision and close vision functions. Due to acquired habits and requirement, only near vision function but not far vision function is used, leading to degradation and reduction of the far vision function, and causing myopia. Looking distantly and looking nearly refer to the fact that a man can see clearly things near and distant, however, this behavior is more abstract, not objectively being reflected. Myopia, regardless of true and false, is only points of congenital and acquired formation. Congenital formation belongs to an organic cause, and requires treatment by other medical device, while 95% of the formation of myopia is acquired, and belongs to functional disorders, ciliary movement is more abstract. Currently, the market has not really effective products that can help users to exercise this function; therefore, the vision cannot be effectively reversed and restored.

### SUMMARY OF THE INVENTION

For the aforementioned deficiencies in the prior art, the prevention proposes a vision correction instrument and a method of correcting vision to regulate ciliary muscle to continuously constrict and relax, thus achieving the purpose of treatment of myopia.

The present invention addresses the aforementioned deficiencies of the prior art presents a vision correction instrument and the correction method to regulate ciliary muscle shrinking relaxation, achieve the purpose of treatment of myopia.

The invention proposes the following technical proposal: a vision correction instrument comprises a display device, a support frame and a keyboard. The display device is provided on the support frame. The display device and the keyboard are in wireless connection. A power button is provided on the display device. A certain distance is provided between the display device and the keyboard.

Furthermore, the support frame comprises a support rod, a cross bar, sliding wheels and a sliding end. Both sides of the support rod are provided with the sliding wheels. The rear side of the cross bar is provided with the sliding end, and the cross bar is connected with the sliding wheels through the sliding end.

Furthermore, a certain distance of 3-5 meters is provided between the display device and the keyboard.

Based on the above vision correction instrument, the invention provides a method of correcting vision, the method comprises the following steps:
starting the display device;
inputting the numbers or letters shown on the display device through the keyboard and pressing an enter key;
repeating the previous step operation;
returning to initial state; and
closing the display device.

Furthermore, before starting the display device, the distance between the keyboard and the display device is adjusted, and the distance between the keyboard and the display device is set to be 3-5 meters.

Furthermore, before starting the display device, a power button on the display device is long pressed and a myopia treatment icon on the display device is clicked.

Furthermore, the method comprises repeating the previous step operation for many times, wherein the numbers or letters shown on the device randomly are repeatedly input into the device. If the previous operation is inputting wrongly, the previous page is turned to and re-entered.

Furthermore, the returning to initial state specifically comprises:
clicking a “Tab” key on the keyboard, and showing a “Restore” button;
clicking a “Enter key” to return to the initial state; and
clicking the “Tab” key through the keyboard and inputting the numbers or letters when an enter box are shown on the display device.

The beneficial effects of the invention are as follows: numbers or letters of the display device appearing randomly are input through the keyboard, they randomly changes, and the font of numbers or letters shown each time becomes sequentially smaller. By displaying the right and left display on the display device and the font size, the adjustment of the front and rear as well as left and right eye can be achieved, making the eye's ciliary muscle constrict and relax, exercising the regulatory function of the ciliary muscle, increasing ciliary activity, slowly recovering the degradation and reduced functionality, and achieving the purpose of treatment of myopia.

### BRIEF DESCRIPTION OF THE DRAWINGS

The exemplary embodiment(s) of the present invention will be understood more fully from the detailed description given below and from the accompanying drawings of various embodiments of the invention, which, however, should not be taken to limit the invention to the specific embodiments, but are for explanation and understanding only.

FIG. 1 is a structure diagram of a vision correction instrument according to the invention; and

FIG. 2 is a structure diagram of a supporting frame of a vision correction instrument according to the invention.

### DETAILED DESCRIPTION OF THE PREFERRED EMBODIMENTS

Exemplary embodiments of the present invention are described herein in the context of vision correction instrument and method of correcting vision.

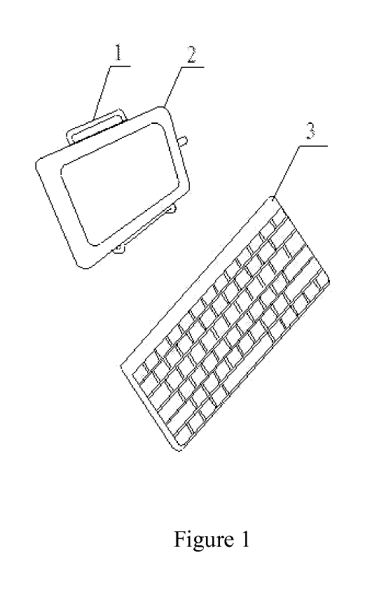

FIG. 1 is a structure diagram of a vision correction instrument according to the invention.

As shown in FIG. 1, a vision correction instrument comprises a display device 1, a support frame 2 and a keyboard 3. The display device 2 is provided on the support frame 1. The display device 2 and the keyboard 3 are in wireless connection. A power button is provided on the display device 2. A certain distance is provided between the display device 2 and the keyboard 3.

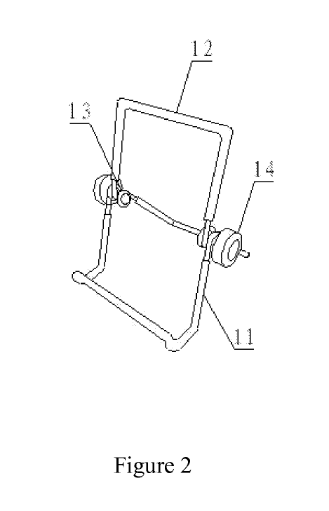

As shown in FIG. 2, FIG. 2 is a structure diagram of a supporting frame of a vision correction instrument according to the invention. The support frame 1 comprises a support rod 11, a cross bar 12, a sliding end 13 and sliding wheels 14. Both sides of the support rod 11 are provided with the sliding wheels 14. The rear side of the cross bar 12 is provided with the sliding end 13, and the cross bar 12 is connected with the sliding wheels 14 through the sliding end 13.

A certain distance of 3-5 meters is provided between the display device 2 and the keyboard 4.

In an example of the invention, the letters input by the keyboard and those shown on the display device have different sizes of fonts, so that the eyes try to see the font, thereby realizing the constriction and relaxation of the ciliary muscle of the eyes, and achieving the purpose of treating myopia.

Based on the above vision correction instrument, the invention provides a method of correcting vision, the method comprises the following steps:
starting the display device;
inputting the numbers or letters shown on the display device through the keyboard and pressing an enter key;
repeating the previous step operation;
returning to initial state; and
closing the display device.

In an example of the invention, the display device is started, the numbers or letters shown on the display device is input through the keyboard and an enter key is pressed. The left and right sides of the display device show large fonts and small fonts respectively, so that the ciliary muscle of the eyes moves in the horizontal direction. During the course that the eyes try to see the numbers or letters, the regulatory function of the ciliary muscle can be exercised, thus enhancing the activity of the ciliary muscle, slowly recovering the degradation and reduced function, and achieving the purpose of reversing and treating myopia.

Before the display device is started, the distance between the keyboard and the display device is adjusted, the distance between the keyboard and the display device is set to be 3-5 meters. The distance between the keyboard and the display device is set to ensure the moderate constriction intensity of the ciliary muscle.

Before the display device is started, a power button on the display device is long pressed and a myopia treatment icon on the display device is clicked. In an example of the invention, a power button is a power switch, the power button is pressed to switch on the power, and the display device is turned on. After the end of use, the power button is pressed to turn off the power, and myopia correction instrument is closed.

In an example of the invention, the numbers or letters shown in the display device are classified based on the font size of a vision chart. The font of the previous level is bigger than that of the next level. During the patient's use, the font is shown step by step through the display device, so that different patients themselves can control and regulate the degree of master, classification can keep track of the extent of the recovery of the patient.

The previous step operation is repeated for many times, wherein the numbers or letters shown on the device randomly are repeatedly input into the device. If the operation is input wrongly, the previous page is returned to and re-enter.

In an example of the invention, by displaying the numbers or letters on the display device, the patient inputs the numbers or letters shown on the display device through the keyboard based on the display on the display device. When the patient inputs the correct numbers or letters, the next level is shown. When the patient inputs numbers or letters wrongly, the previous display is automatically return and he/she re-enters. Since the numbers or letters shown on the display device are randomly changed, the patient cannot enter based on his/her memory, he/she must always be able to see them clearly with their eyes and by his/her efforts to promote the patient's eye ciliary muscle to have movement and changes, thus enhancing the activity of the ciliary muscle.

The returning to initial state specifically comprises:
clicking a “Tab” key on the keyboard, and showing a “Restore” button;
clicking a “Enter key” to return to the initial state; and
clicking the “Tab” key on the keyboard and inputting the numbers or letters when an enter box shows on the display device.

In an example of the invention, in the process of using this vision correction instrument, the patient can always return to the original state, and choose the font size. The initial state of the invention is set to be the largest-font display state, and the font entered in turn is decreased gradually. In the process of entering, the Tab key on the keyboard is clicked, the display device shows a blue button, the Enter key is pressed to return to the initial state of a large font, and the Tab key is clicked again, an input box is shown and numbers and letters are input.

The display device of the invention is in the left and right display, wherein the left side of the display device shows big numbers or letters while the right side of the display device shows small numbers or letters.

An example of the invention classifies the fonts and sizes of the letters shown in the display device, the fonts of the numbers or letters input each time is smaller than those input previously. When in use, selection can be carried out according to needed vision correction case, making the correction process be suitable for the patient, so that different patients can control and master the regulation degree according to their needs, the classification can keep abreast of patient recovery.

When the numbers or letters are shown on the display device, a display device shows the different sizes of the fonts at the left and right sides to form the four directions of the distance adjustment range of the eyes including the left, the right, the distant and the near. Ciliary muscle can continuously carry out relaxation and constriction to adjust the changes of the lens, alleviate eye fatigue, and achieve the purpose of treatment of myopia.

As the eye is passive when seeing the material, through the instrument, the continuous changes in the distances and sizes can help us independently move the ciliary muscle. By the changes in the distance and size, the patient can achieve the strength and frequency meeting the requirements. As the ciliary muscle continuously constricts and relaxes, ciliary activity will increase, and the rigid and lost functions will be recovered, thus achieving the desired effect of therapeutic and reversal of myopia.

A vision correction instrument provided in the invention can exercise and correct the functions of ciliary muscle and restore the elasticity of ciliary muscle. Distance changes and the ciliary muscle continuously constrict and relax. During the use process, it is a normal phenomenon if the eyes are discomfort, soreness and astringent. Because it indicates that the ciliary muscle functions of a child are being exercising, strengthening and recovering.

While particular embodiments of the present invention have been shown and described, it will be obvious to those skilled in the art that, based upon the teachings herein, changes and modifications may be made without departing from this invention and its broader aspects. Therefore, the appended claims are intended to encompass within their scope of all such changes and modifications as are within the true spirit and scope of the exemplary embodiment(s) of the present invention.

What is claimed is:

1. A vision correction instrument comprises a display device, a support frame and a keyboard,
wherein the display device is provided on the support frame;
the display device and the keyboard are in wireless connection;
a power button is provided on the display device; and
a certain distance is provided between the display device and the keyboard.

2. The vision correction instrument according to claim 1, wherein the support frame comprises a support rod, a cross bar, sliding wheels and a sliding end; both sides of the support rod are provided with the sliding wheels; the rear side of the cross bar is provided with the sliding end; and the cross bar is connected with the sliding wheels through the sliding end.

3. The vision correction instrument according to claim 1, wherein a certain distance of 3-5 meters is provided between the display device and the keyboard.

4. A method of correcting vision provided by a vision correction instrument according to claim 1, the method comprising the following steps:
starting the display device;
inputting the numbers or letters shown on the display device through the keyboard and pressing an enter key;
repeating the previous step operation;
returning to an initial state; and
closing the display device.

5. The method of correcting vision according to claim 4, further comprising: adjusting a distance between the keyboard and the display device, and setting the distance between the keyboard and the display device to be 3-5 meters before starting the display device.

6. The method of correcting vision according to claim 4, further comprising: long pressing a power button on the display device and clicking a myopia treatment icon on the display device before starting the display device.

7. The method of correcting vision according to claim 4, further comprising: repeating the previous step operation for many times, wherein the numbers or letters shown on the device randomly are repeatedly input into the device, if inputting wrongly, returning to the previous page and re-entering.

8. The method of correcting vision according to claim 4, wherein the returning to the initial state thither comprises:
clicking a “Tab” key on the keyboard, and displaying a “Restore” button;
clicking a “Enter key” to return to the initial state; and
clicking the “Tab” key through the keyboard and inputting the numbers or letters when an enter box are shown on the display device.
